(19) United States
(12) Patent Application Publication
(10) Pub. No.: US 2024/0371141 A1
(43) Pub. Date: Nov. 7, 2024

(54) METHODS AND APPARATUS PROVIDING TRAINING UPDATES IN AUTOMATED DIAGNOSTIC SYSTEMS

(71) Applicant: Siemens Healthcare Diagnostics Inc., Tarrytown, NY (US)

(72) Inventors: Venkatesh NarasimhaMurthy, Hillsborough, NJ (US); Vivek Singh, Princeton, NJ (US); Yao-Jen Chang, Princeton, NJ (US); Benjamin S. Pollack, Jersey City, NJ (US); Ankur Kapoor, Plainsboro, NJ (US); Rayal Raj Prasad Nalam Venkat, Princeton, NJ (US)

(73) Assignee: Siemens Healthcare Diagnostics Inc., Tarrytown, NY (US)

(21) Appl. No.: 18/576,262

(22) PCT Filed: Jul. 6, 2022
(86) PCT No.: PCT/US2022/073474

§ 371 Date: Jan. 3, 2024

Related U.S. Application Data
(60) Provisional application No. 63/219,343, filed on Jul. 7, 2021.

(51) Int. Cl. G06V 10/774; G01N 33/49; G06T 7/00; G06V 10/26; G06V 10/56; G06V 10/776; G16H 10/40; G16H 50/20

(52) U.S. Cl. CPC G06V 10/774; G01N 33/491; G06T 7/0012; G06V 10/26; G06V 10/56; G06V 10/776; G16H 10/40; G16H 50/20; G06T 2207/20081; G06V 2201/03

(57) ABSTRACT

A method of characterizing a specimen container or a specimen in an automated diagnostic system includes capturing an image of a specimen container containing a specimen using an imaging device. The method further includes characterizing the image using a first AI model and determining whether a characterization confidence of the image is below a pre-selected threshold. The first AI model is retrained with the image having the characterization confidence below the pre-selected threshold to a second AI model, wherein the retraining includes data selected from one or more of a group of: non-image data, and text data. Quality check modules and systems configured to perform the method are also described, as are other aspects.

### CROSS REFERENCE TO RELATED APPLICATION

This application claims the benefit of U.S. Provisional Patent Application No. 63/219,343, entitled “METHODS AND APPARATUS PROVIDING TRAINING UPDATES IN AUTOMATED DIAGNOSTIC SYSTEMS” filed Jul. 7, 2021, the disclosure of which is hereby incorporated by reference in its entirety for all purposes.

### FIELD

Embodiments of this disclosure relate to methods and apparatus configured to provide training in automated diagnostic systems.

### BACKGROUND

Automated diagnostic systems analyze (e.g., test) biological specimens, such as whole blood, blood serum, blood plasma, urine, interstitial liquid, cerebrospinal liquid, and the like, in order to identify analytes or other constituents in the specimens. The specimens are usually contained within specimen containers (e.g., specimen collection tubes) that can be transported via automated track systems to various pre-processing modules, pre-screening modules, and analyzers (e.g., including immunoassay and clinical chemistry) within such automated diagnostic systems.

In some systems, the pre-processing modules can carry out processing on the specimen or specimen container, such as de-sealing, centrifugation, aliquoting, and the like, all prior to analysis by one or more analyzers. In some systems, the pre-screening may be used to characterize specimen containers and/or the specimens. Characterization may be performed by an artificial intelligence (AI) model and may include a segmentation operation, which may identify various regions of the specimen containers and/or specimens. Characterization of the specimens using the AI model may include an HILN process that determines a presence of an interferent, such as hemolysis (H), icterus (I), and/or lipemia (I), in a specimen to be analyzed or determining that the specimen is normal (N) and can thus be further processed.

After pre-processing and/or pre-screening, the specimens are analyzed by one or more analyzers of the automated diagnostic system. Measurements may be performed on the specimens via photometric analyses such as, fluorometric absorption and/or emission analyses. Other measurements may be used. The measurements may be analyzed to determine amounts of analytes or other constituents in the specimens.

Over time, components of the systems may change. For example, imaging devices and illumination sources used during imaging may change. In some embodiments, the specimen containers may also change over time. The AI model(s) may not be adequately trained to characterize the components and specimen containers that have changed over time. Thus, the above-described analysis using the AI models may be erroneous.

Based on the foregoing, improved methods of training AI models for use in automated diagnostic systems are sought.

### SUMMARY

According to a first aspect, a method of characterizing a specimen container or a specimen in an automated diagnostic system is provided. The method includes capturing an image of a specimen container containing a specimen using an imaging device; characterizing the image using a first AI model; determining whether a characterization confidence of the image is below a pre-selected threshold; and retraining the first AI model with at least the image having the characterization confidence below the pre-selected threshold to a second AI model, wherein the retraining includes data selected from one or more of a group of: non-image data and text data.

According to another aspect, a method of characterizing a specimen in an automated diagnostic system is provided. The method includes capturing an image of the specimen using an imaging device; characterizing the image using a first AI model to determine a presence of at least one of hemolysis, icterus, or lipemia; determining whether a characterization confidence of the determination of the presence of at least one of hemolysis, icterus, or lipemia is below a pre-selected threshold; and retraining the first AI model with at least the image having the characterization confidence below the pre-selected threshold to a second AI model, wherein the retraining includes data selected from one or more of a group of: non-image data, and text data.

According to another aspect, an automated diagnostic system is provided. The automated diagnostic system includes an imaging device configured to capture an image of a specimen container containing a specimen; and a computer configured to: characterize the image using a first AI model; determining whether a characterization confidence of the image is below a pre-selected threshold; and retrain the first AI model with at least the image having the characterization confidence below the pre-selected threshold to a second AI model, wherein the retraining includes data selected from one or more of a group of: non-image data and text data.

Still other aspects, features, and advantages of this disclosure may be readily apparent from the following description and illustration of a number of example embodiments and implementations, including the best mode contemplated for carrying out the invention. This disclosure may also be capable of other and different embodiments, and its several details may be modified in various respects, all without departing from the scope of the disclosure. This disclosure is intended to cover all modifications, equivalents, and alternatives falling within the scope of the claims.

### BRIEF DESCRIPTION OF THE DRAWINGS

The drawings, described below, are for illustrative purposes, and are not necessarily drawn to scale. Accordingly, the drawings and descriptions thereof are to be regarded as illustrative in nature, and not as restrictive. The drawings are not intended to limit the scope of the disclosure in any way.

FIG. 1 illustrates a top schematic view of an automated diagnostic system including one or more modules and one or more instruments configured to analyze specimen containers and/or specimens according to one or more embodiments.

FIG. 2A illustrates a side view of a specimen container including a separated specimen with a serum or plasma portion that may contain an interferent according to one or more embodiments.

FIG. 2B illustrates a side view of the specimen container of FIG. 2A held in an upright orientation in a holder that can be transported within the automated diagnostic system of FIG. 1 according to one or more embodiments.

FIG. 3 is a flowchart describing a method of training an artificial intelligence model to analyze specimens and/or specimen containers in automated diagnostic systems according to one or more embodiments.

FIG. 4A illustrates a schematic top view of a quality check module (with top removed) including multiple viewpoints and configured to capture and analyze multiple images to enable a characterization such as segmentation and/or pre-screening for HILN according to one or more embodiments.

FIG. 4B illustrates a schematic side view of the quality check module (with enclosure wall removed) of FIG. 4A taken along section line 4B-4B of FIG. 4A according to one or more embodiments.

FIG. 5 illustrates a functional block diagram of an HILN network configured to perform segmentation and interferent determinations of a specimen in a specimen container while providing training updates to the HILN network based on characterization performance according to one or more embodiments.

FIG. 6 illustrates a flowchart showing a method of characterizing a specimen container or a specimen in an automated diagnostic system according to one or more embodiments.

FIG. 7 illustrates a flowchart showing a method of characterizing a specimen in an automated diagnostic system according to one or more embodiments.

### DETAILED DESCRIPTION

Automated diagnostic systems described herein analyze (e.g., test) biological specimens to determine the presence and/or concentrations of analytes in the specimens. In some embodiments, the systems may perform one or more pre-screening analyses on the specimens. In some embodiments, the systems may perform pre-screening analyses on specimen containers. The analyses may be performed using artificial intelligence (AI) models as described herein.

The AI models described herein may be implemented as machine learning, neural networks, and other AI algorithms. The AI models may be trained to characterize images or portions of captured images. Characterizing images includes identifying items in one or more portions of an image. For example, a first or initial AI model may be trained to characterize items in images that are expected to be captured by the system, such as specimens and specimen containers. In some embodiments, a large dataset of images of items that are to be characterized may be captured in different configurations, such as different views and/or different lighting conditions, and may be used to train a first AI model. One or more algorithms or programs may be used to check characterization confidences that trained the first AI model. In some embodiments, the characterization confidences may be in the form of a value (e.g., between 1 and 100) or a percentage. A low characterization confidence may be indicative of inadequate characterization, i.e., the AI model has not been adequately trained to recognize the specimen or specimen container.

Over time, the items being characterized may change. In some embodiments, the conditions under which the images are captured also may change. These changed items and conditions may be due to hardware changes (e.g., updates), software changes, changes in specimen containers, labeling changes on the specimen containers, changes in assays, and other changes. The first AI model may not be able to characterize these changed items or may not be able to characterize the items under the changed conditions. For example, images used to train the first AI model may not include every variation of a specimen container and/or a specimen received in a system. For example, the sizes, types, and characteristics of specimen containers may change over time, resulting in incorrect or low characterization confidences. Accordingly, the first AI model may have to be updated to a second AI model in order to be able to properly characterize the changed items and conditions.

Conventional Automated diagnostic systems do not provide for easy and/or automatic updates to AI models. Rather, the process for updating AI models may be relatively cumbersome. For example, in some embodiments, the deficiencies in the first AI model may not be identified until the system fails. Troubleshooting the system failure may be required to determine that the first AI model is not adequate. Once the first AI model inadequacies are identified, data pertaining to the above-described changes is collected and transmitted to engineering teams. This data is then used by the engineering teams to update or retrain the first AI model. The conventional update processes are very expensive and time consuming. Methods and apparatus disclosed herein provide for improved updating and/or replacing of first AI models with second AI models.

The AI models described herein may be used in systems that include a quality check module. A quality check module performs pre-screening of specimens and/or specimen containers based on images captured in the quality check module. The prescreening may use an AI model as described herein to characterize images of specimen containers and/or specimens. The pre-screening characterization may include performing segmentation and/or interferent (e.g., HILN—hemolytic, icteric, lipemic, normal) identifications on the captured images. The segmentation determination may identify various regions (areas) in the image of the specimen container and specimen, such as a serum or plasma portion, a settled blood portion, a gel separator (if used), an air region, one or more label regions, a type of specimen container (indicating, e.g., height and width or diameter), and/or a type and/or color of a specimen container cap.

As described above, the interferent identified by the AI models may include hemolysis (H), icterus (I), or lipemia (L). The degree of hemolysis may be quantified using the AI model by assigning a hemolytic index (e.g., H0-H6 in some embodiments and more or less in other embodiments). The degree of icterus may be quantified using the AI model by assigning an icteric index (e.g., I0-I6 in some embodiments and more or less in other embodiments). The degree of lipemia may be quantified using the AI model by assigning a lipemic index (e.g., L0-L4 in some embodiments and more or less in other embodiments). In some embodiments, the pre-screening process may include determination of an un-centrifuged (U) class for a serum or plasma portion of a specimen that has not been centrifuged.

An HILN network implemented by an AI model may be or include a segmentation convolutional neural network (SCNN) that receives as input one or more captured images of fractionated specimens contained in specimen containers. The SCNN may include, in some embodiments, greater than 100 operational layers including, e.g., BatchNorm, ReLU activation, convolution (e.g., 2D), dropout, and deconvolution (e.g., 2D) layers to extract features, such as simple edges, texture, and parts of the serum or plasma portion and label-containing regions of images. Top layers, such as fully convolutional layers, may be used to provide correlation between the features. The output of the layer may be fed to a SoftMax layer, which produces an output on a per pixel (or per superpixel (patch) —including n×n pixels) basis concerning whether each pixel or patch includes HIL or is normal.

In some embodiments, only an output of HILN may be provided by the SCNN. In other embodiments, the output of the SCNN may include multiple classes of HILN, such as greater than 20 classes of HILN, so that for each interferent present, an estimate of the level (index) of the interferent can also be obtained. Other numbers of classes of each of HIL may be included in the SCNN. The SCNN may also include a front-end container segmentation network (CSN) to determine a specimen container type and a specimen container boundary. Other types of HILN networks may be used in quality check modules.

In some embodiments, an initial set of training images used to train an initial or first AI model may be compiled by operating a newly-installed automated diagnostic analysis system for a given period of time (e.g., one or two weeks). Captured image data of specimens received in the newly installed system may be forwarded to a database/server (which may be local and/or a part of the newly installed system or it may be a cloud-based server). The image data may be annotated (e.g., annotated manually and/or annotations generated automatically) to create the initial set of annotated training images. The set of annotated training images may then be used to train the initial or first AI model in the HILN network of the quality check module of the automated diagnostic analysis system.

During operation of the automated diagnostic analysis system, images of specimens having characterizations generated by the HILN network that are determined to be incorrect or have low characterization confidences (e.g., low confidence levels) may not be automatically forwarded to an analyzer of the automated diagnostic analysis system, but may be stored for further review. For example, the images of specimens having characterizations determined to be incorrect or having low characterization confidences may be stored (and encrypted in some embodiments) in a database/server. The training updates (e.g., training of the second AI model) may be based at least in part on the incorrect or low confidence characterizations that stored in the database/server.

In some embodiments, the training updates may be based at least in part on manual annotations and/or automatically generated annotations of the captured images of the specimens having characterizations determined to be incorrect or have low confidence. The training updates may be forwarded to the HILN network for incorporation therein via retraining of the first AI model to generate a retrained second AI model. In some embodiments, a report or prompt of the availability of one or more training updates may be provided to a user to allow the user to decide when and if the training updates are to be incorporated into the HILN network. In other embodiments, training updates may be automatic.

In some embodiments, the initial set of training images and/or the training updates, each of which is software based, may be provided to an automated diagnostic analysis system (and the HILN network in particular) as a retrained model via the internet or by using a physical media (e.g., a storage device containing programming instructions and data).

Some embodiments of systems disclosed herein can provide continuous training updates of AI models that may be automatically incorporated into the system via retraining and/or AI model replacement on a frequent or regular basis, such as, e.g., upon meeting or exceeding a threshold number of incorrect or low characterization confidences. Other criteria may be used to automatically incorporate training updates into the systems. In some embodiments, training updates may be incorporated into a system by a user at the discretion of the user, such as via a user prompt.

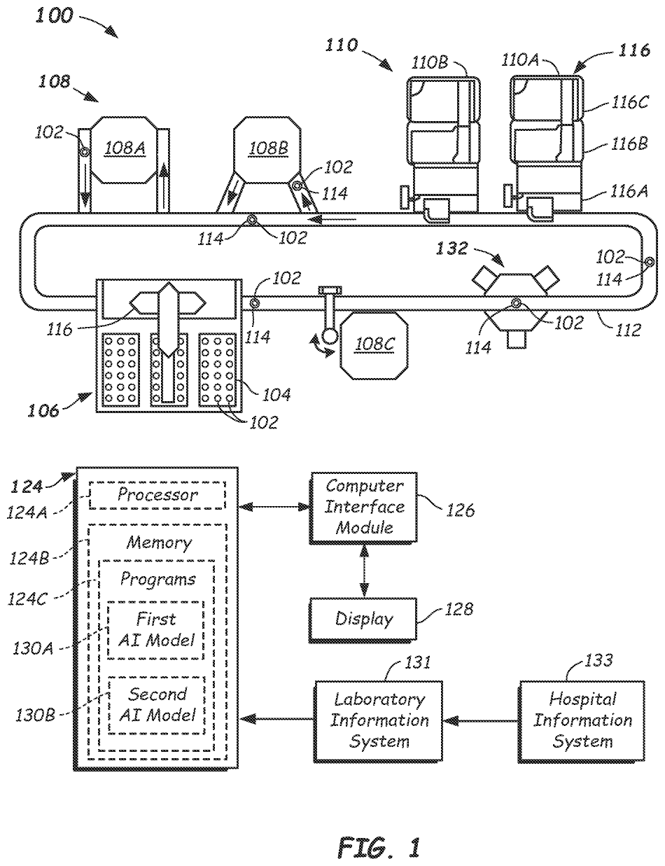

Further details of characterization apparatus and methods including updating AI models will be further described with reference to FIGS. 1-7 herein.

Reference is now made to FIG. 1, which illustrates an example embodiment of an automated diagnostic system 100 configured to process and/or analyze biological specimens stored in specimen containers 102. The specimen containers 102 may be any suitable containers, including transparent or translucent containers, such as a blood collection tubes, test tubes, sample cups, cuvettes, or other containers capable of containing and allowing imaging of the specimens contained therein. The specimen containers 102 may be varied in size and may have different cap colors and/or cap types.

The specimen containers 102 may be received at the system 100 in one or more racks 104 provided at a loading area 106. The specimen containers 102 may be transported throughout the system 100, such as to and from modules 108 and instruments 110 on a track 112 by carriers 114.

Processing of the specimens and/or the specimen containers 102 may include preprocessing or pre-screening of the specimens and/or the specimen containers 102 prior to analysis by one or more of the modules 108 configured as analyzer modules, which may be referred to herein as analyzers. The system 100 may also include one or more instruments 110, wherein each instrument may include one or more modules, such as preprocessing modules and/or analyzer modules. In the embodiment of FIG. 1, the system 100 includes a first instrument 110A and a second instrument 110B that may each include a plurality of modules. The first instrument 110A, which may be similar or identical to the second instrument 110B, includes three modules 116 that may perform functions similar to or identical to the modules 108 as described herein. The modules 116 are referred to individually as a first module 116A, a second module 116B, and a third module 116C. The instruments 110 may include other numbers of modules.

In some embodiments, the first module 116A may be a preprocessing module, for example, that processes the specimen containers 102 and/or specimens located therein prior to analyses by analyzer modules. The second module 116B and the third module 116C may be analyzer modules that analyze specimens as described herein. Other embodiments of the instruments 110 may be used for other purposes in the system 100.

In the embodiment of FIG. 1, the system 100 includes a plurality of modules, including a first module 108A, a second module 108B, and a third module 108C. Other modules that perform specific functions and/or processes are provided and described independent of the first module 108A, the second module 108B, and the third module 108C. At least one of the modules 108 may perform preprocessing functions and may include a decapper and/or a centrifuge, for example. In some embodiments, one or more of the modules 108 may be a clinical chemistry analyzer and/or an assaying instrument, or the like, or combinations thereof. More or fewer modules 108 and instruments 110 may be used in the system 100.

The modules implemented as analyzer modules of the modules 108 and the instruments 110 may be any combination of any number of clinical chemistry analyzers, assaying instruments, and/or the like. The term “analyzer” as used herein includes a device used to analyze a specimen for chemistry or to assay for the presence of, amount, or functional activity of a target entity (e.g., an analyte), such as DNA or RNA, for example. Analytes commonly tested for in analyzer modules include enzymes, substrates, electrolytes, specific proteins, drugs of abuse, and therapeutic drugs.

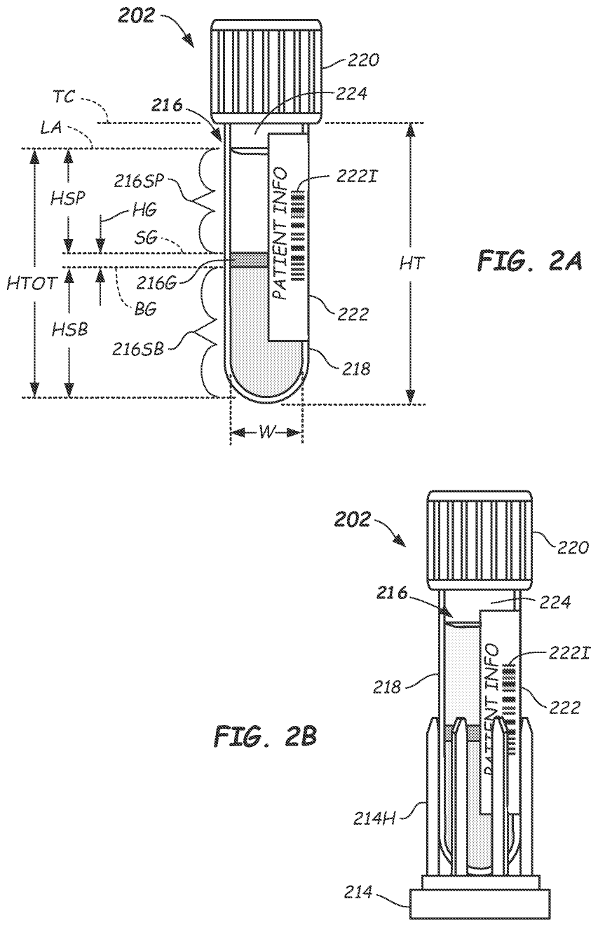

Additional reference is made to FIGS. 2A-2B, which illustrates an embodiment of a specimen container 202 with a specimen 216 located therein. The specimen container 202 may be representative of the specimen containers 102 (FIG. 1) and the specimen 216 may be representative of specimens located in the specimen containers 102. The specimen container 202 may include a tube 218 and may be capped with a cap 220. Caps on different specimen containers may be of different types and/or colors (e.g., red, royal blue, light blue, green, grey, tan, yellow, or color combinations), which may have meaning in terms of specific tests the specimen container 202 is used for, the type of additive included therein, whether the specimen container includes a gel separator, or the like. In some embodiments, the cap type may be determined by a characterization method described herein.

The specimen container 202 may be provided with at least one label 222 that may include identification information 222I (i.e., indicia) thereon, such as a barcode, alphabetic characters, numeric characters, or combinations thereof. The identification information 222I may include or be associated data provided by a laboratory information system (e.g., LIS 131—FIG. 1), such as a database in the LIS 131. The database may include information referred to as text data such as patient information, including name, date of birth, address, and/or other personal information as described herein. The database may also include other text data, such as tests to be performed on the specimen 216, time and date the specimen 216 was obtained, medical facility information, and/or tracking and routing information. Other text data may also be included. The data in the LIS 131 may be received from a hospital information system 133 that receives test orders and the like from medical providers.

The identification information 222I may be darker (e.g., black) than the label material (e.g., white paper) so that the identification information 222I can be readily imaged. The identification information 222I may indicate, or may otherwise be correlated, via the LIS or other test ordering system, to a patient's identification as well as tests to be performed on the specimen 216. The identification information 222I may be provided on the label 222, which may be adhered to or otherwise provided on an outside surface of the tube 218. In some embodiments, the label 222 may not extend all the way around the specimen container 202 or along a full length of the specimen container 202.

The specimen 216 may include a serum or plasma portion 216SP and a settled blood portion 216SB contained within the tube 218. A gel separator 216G may be located between the serum or plasma portion 216SP and the settled blood portion 216SB. Air 224 may be provided above the serum and plasma portion 216SP. A line of demarcation between the serum or plasma portion 216SP and the air 224 is defined as the liquid-air interface (LA). A line of demarcation between the serum or plasma portion 216SP and the gel separator is defined as a serum-gel interface (SG). A line of demarcation between the settled blood portion 216SB and the gel separator 216G is defined as a blood-gel interface (BG). An interface between the air 224 and the cap 220 is defined as a tube-cap interface (TC).

The height of the tube (HT) is defined as a height from a bottom-most part of the tube 218 to a bottom of the cap 220 and may be used for determining tube size (e.g., tube height and/or tube volume). A height of the serum or plasma portion 216SP is HSP and is defined as a height from a top of the serum or plasma portion 216SP at LA to a top of the gel separator 216G at SG. A height of the gel separator 216G is HG and is defined as a height between SG and BG. A height of the settled blood portion 216SB is HSB and is defined as a height from the bottom of the gel separator 216G at BG to a bottom of the settled blood portion 216SB. HTOT is a total height of the specimen 216 and equals the sum of HSP, HG, and HSB. The width of the cylindrical portion of the inside of the tube 218 is W. Preprocessing performed in one or more of the preprocessing modules 108 and/or instruments 110 may measure or calculate at least one of the above-described dimensions.

The embodiment of FIG. 2B illustrates a side elevation view of the specimen container 202 located in a carrier 214. The carrier 214 may be representative of the carriers 114 (FIG. 1). The carrier 214 may include a holder 214H configured to hold the specimen container 202 in a defined upright position and orientation. The holder 214H may include a plurality of fingers or leaf springs that secure the specimen container 202 in the carrier 214, but some may be moveable or flexible to accommodate different sizes (widths) of the specimen container 202. In some embodiments, the carrier 214 may leave from the loading area 106 (FIG. 1) after being offloaded from the one or more of the racks 104.

Referring again to FIG. 1, the system 100 may include a computer 124 or be configured to communicate with an external computer. The computer 124 may be a microprocessor-based central processing unit CPU with suitable memory, software, and conditioning electronics and drivers for operating the various components, modules 108, and instruments 110 of the system 100. The computer 124 may include a processor 124A and memory 124B, wherein the processor 124A is configured to execute programs 124C stored in the memory 124B. The computer 124 may be housed as part of, or separate from, the system 100. The programs 124C may operate components of the system 100 and may perform characterizations as described herein.

The computer 124, by way of the programs 124C, may control movement of the carriers 114 to and from the loading area 106, about the track 112, to and from the modules 108 and the instruments 110, and to and from other modules and components of the system 100. One or more of the modules 108 or instruments 110 may be in communications with the computer 124 through a network, such as a local area network (LAN), wide area network (WAN), or other suitable communication network, including wired and wireless networks. In some embodiments, the operation of some or all of the above-described modules 108 and/or instruments 110 may be performed by the computer 124.

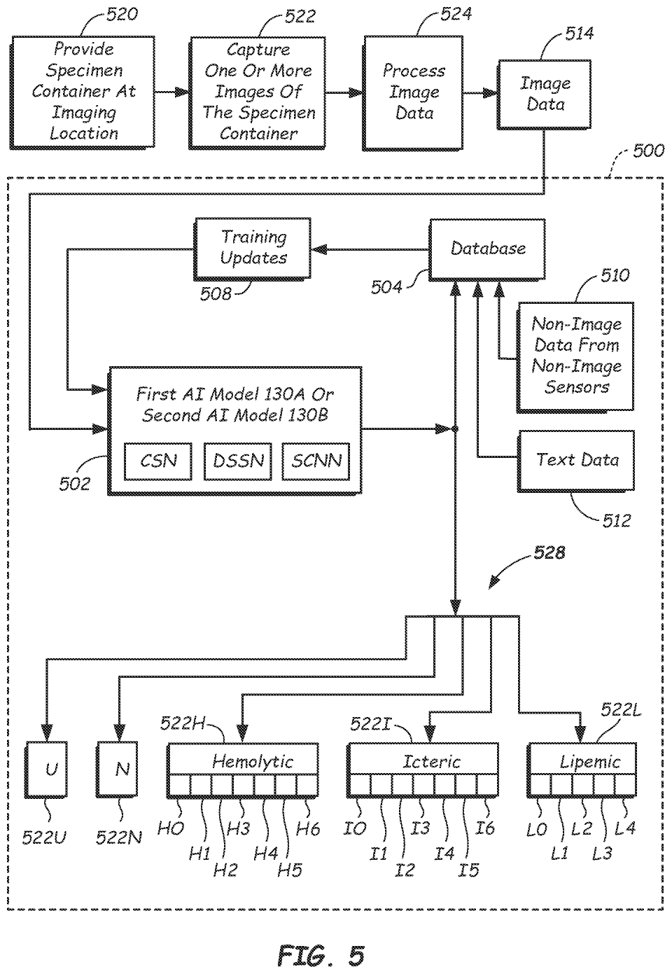

One or more of the programs 124C may be artificial intelligence (AI) models or algorithms that process and/or analyze image data and other data as described herein. The other data may include non-image data (510—FIG. 5) and text data (512—FIG. 5), for example. In the embodiment of FIG. 1, the memory 124B is shown as storing a first AI model 130A and a second AI model 130B. The first AI model 130A references the AI model that has not been updated or replaced as described herein. For example, a first AI model initially provided with the system 100 may be the first AI model 130A. The second AI model 130B is the first AI model 130A as updated and/or replaced with the second AI model 130B as described herein. In some embodiments, the second AI model 130B may be an update or replacement of the first AI model 130A regardless of whether the first AI model 130A was the initial AI model.

The first AI model 130A and the second AI model 130B may be implemented as one or more of the programs 124C or an algorithm that is stored in the memory 124B and executed by the processor 124A. In some embodiments, the first AI model 130A and the second AI model 130B may be executed remotely from the system 100. The first AI model 130A and the second AI model 130B may be implemented as various forms of artificial intelligence, including, but not limited to, neural networks, including convolutional neural networks (CNNs), deep learning, regenerative networks, and other machine learning and artificial intelligence algorithms. Accordingly, the first AI model 130A and the second AI model 130B may not be simple lookup tables. Rather, the first AI model 130A and the second AI model 130B are trained to recognize (e.g., characterize) a variety of different images. A lookup table, on the other hand, is only able to identify images that are specifically in the lookup table.

In some embodiments, the computer 124 may be coupled to a computer interface module (CIM) 126. The CIM 126 and/or the computer 124 may be coupled to a display 128. The CIM 126, in conjunction with the display 128, enables a user to access a variety of control and status display screens and to input data into the computer 124. These control and status display screens may display and enable control of some or all aspects of the modules 108 and/or instruments 110 used for preparation, pre-screening, and analysis of specimen containers 102 and/or the specimens located therein. Thus, the CIM 126 may be adapted to facilitate interactions between a user and the system 100. The display 128 may be configured to display a menu including icons, scroll bars, boxes, and buttons through which the operator may interface with the system 100. The menu may include a number of functional elements programmed to display and/or operate functional aspects of the system 100. In some embodiments, the display 128 may include a graphical user interface that enables a user to instruct the computer 124 to update the first AI model 130A as described herein.

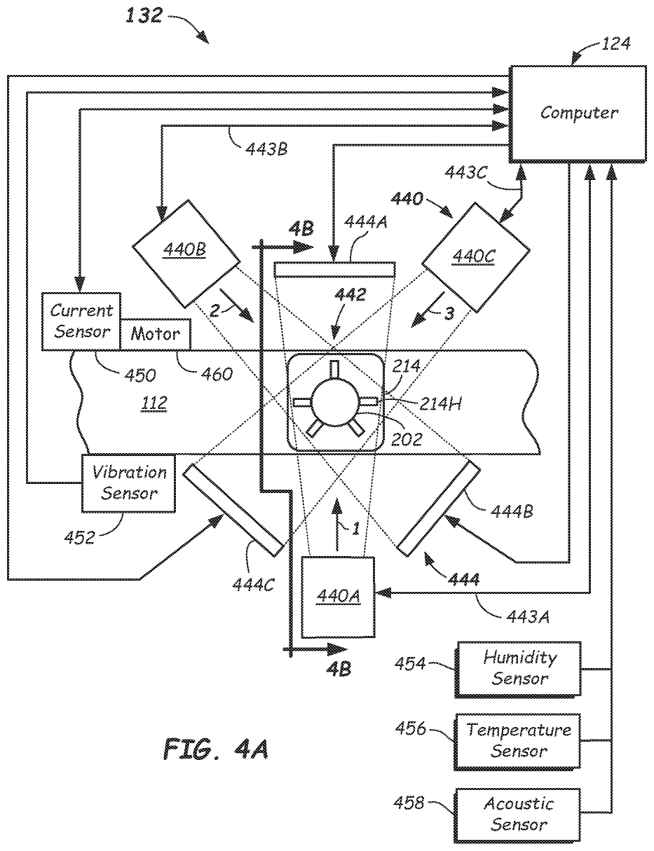

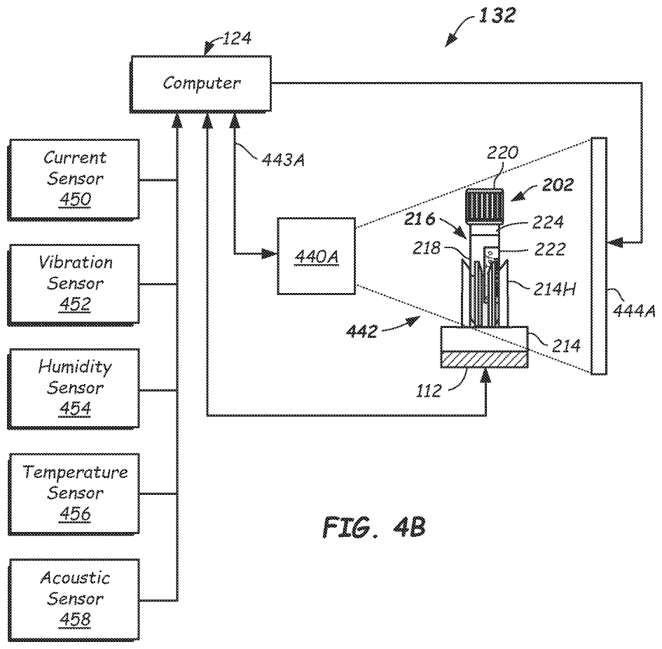

As described herein, the modules 108 and the instruments 110 may perform analyses on the specimen containers 102 and/or the specimens (e.g., specimen 216—FIGS. 2A-2B) located in the specimen containers 102. In some embodiments described in greater detail herein, the analyses may be performed by photometric analysis using the first AI model 130A or the second AI model 130B. For example, images of the specimen containers 102 and/or the specimens located in the specimen containers 102 may be captured by imaging devices (e.g., imaging devices 440—FIG. 4A). The captured images are in the form of image data that may be processed by the programs 124C executed on the computer 124. For example, the first AI model 130A or the second AI model 130B may process the image data as described herein.

In some embodiments, specimens and/or specimen containers 102 are front illuminated in one or more of the modules 108 and/or the instruments 110. Images of the reflected light from the specimen containers 102 and/or the specimens are captured by one or more imaging devices and converted to image data that is processed as described herein. In some embodiments, images of light transmitted through the specimens and/or the specimen containers 102 is captured and converted to image data that is processed as described herein. In some embodiments, chemicals are added to the specimens to cause the specimens to fluoresce and emit light under certain conditions. Images of the emitted light may be captured and converted to image data that is processed as described herein.

The first AI model 130A may be trained by a first validation dataset. The first validation dataset is data collected and used to train and/or verify the first AI model 130A. In some embodiments, the first validation dataset may include data that is verified by various testing or analyses mechanisms. In some embodiments, the first validation dataset may include data that was used for regulatory approval of the system 100 and/or similar systems. For example, in some embodiments, the first validation dataset may include data that may be collected across multiple systems that may be identical or similar to the system 100. In some embodiments, the first validation dataset may be compressed and/or encrypted. In some embodiments, the first validation dataset and/or the first AI model 130A and the second AI model 130B may be stored and/or executed remotely, such as in a cloud. The ground truth for the first validation data set may come from secondary resources, such as a gold standard device and/or data based on the gold standard device. In other embodiments, the gold truth may be automatically generated using an existing trained system or by self-supervision.

Over time, changes, in the data processed by the system 100, including, for example, data generated by the modules 108 and/or the instruments 110, may occur. These changes include, for example, hardware changes (e.g., updates), software changes, changes in the specimen containers 102, changes in the labels (e.g., label 222) and/or barcodes (e.g., information 2201) affixed to the specimen containers 102, assay protocols, and other changes. These changes may not be able to be characterized (e.g., identified) by the first AI model 130A, so the system 100 may have to be updated to a new AI model (e.g., the second AI model 130B). The methods of updating the system 100 to the second AI model 130B are described herein.

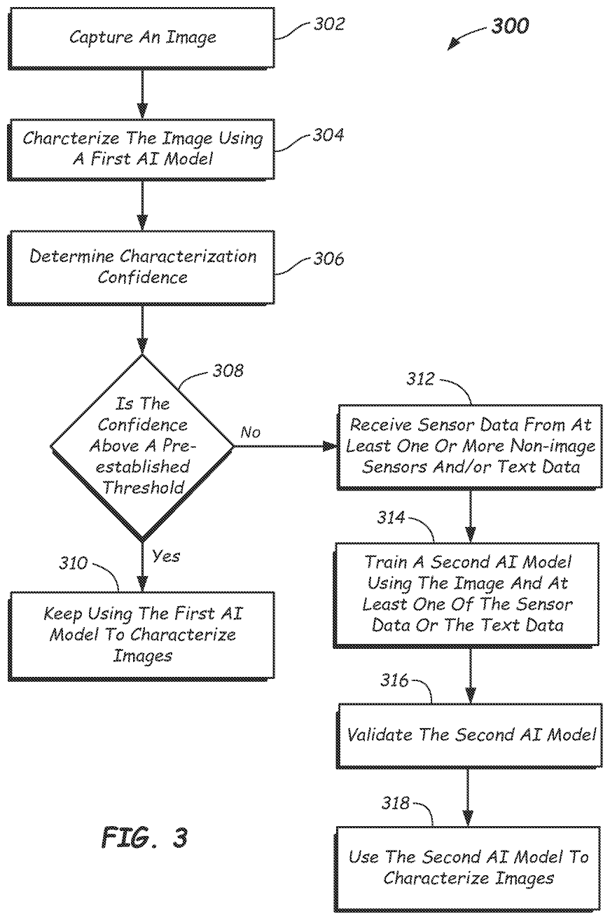

Additional reference is made to FIG. 3, which illustrates a flowchart showing a method 300 that includes updating the system 100 from the first AI model 130A to the second AI model 130B. The method 300 also illustrates using an artificial intelligence model (e.g., first AI model 130A and the second AI model 130B) to analyze specimens (e.g., specimen 216) and/or specimen containers (e.g., specimen container 202) in automated diagnostic systems (e.g., system 100) according to one or more embodiments.

The method 300, in 302, includes capturing an image. The image may be captured using an imaging device (e.g., one or more of the imaging devices 440, FIG. 4A) located in one or more of the modules 108 or the instruments 110. The captured image may be converted to image data so that the captured image may be processed by the programs 124C. For example, the image data may be in a form that enables the first AI model 130A and/or the second AI model 130B to analyze (e.g., characterize) the image data as described herein.

The method 300 includes, in 304, characterizing the image using the first AI model 130A. Different characterizations are described in greater detail. In some embodiments, the characterization may include identifying one or more items in the captured image, such as the specimen container 202 (FIGS. 2A-2B) or the specimen 216 (FIGS. 2A-2B) located in the specimen container 202. The characterization may, as examples, identify the cap 220, including the type and color of the cap 220, the heights of the tube 218 and heights of the portions of the specimen 216 described in FIG. 2A, and characteristics of the specimen 216 as described in greater detail herein.

The method 300, in 306, includes determining confidence of the characterization (characterization confidence) performed in 304. The characterization confidence may be a score or probability that the first AI model 130A characterized or correctly identified items in the captured image. Various known techniques may be used to determine the characterization confidence as described herein. In some embodiments, the characterization confidence may be zero if the characterization was not able to characterize or identify one or more items in the captured image.

The method 300 includes, in 308, determining whether the characterization confidence is above a pre-established threshold. If the confidence is above the pre-established threshold, processing proceeds to 308 where the first AI model 130A is used to characterize the captured image and future captured images. In some embodiments, the pre-established threshold may be 0.7 on a scale between zero and 1.0. This pre-established threshold provides a likelihood that the characterization is correct. In other embodiments, the pre-established threshold may be 0.9 on a scale between zero and 1.0. This pre-established threshold provides more confidence that the characterization is correct.

If, in 308, the determination is made that the confidence is not above the pre-selected threshold, the system or other device generates the second AI model 130B (FIG. 1) as described herein. In some embodiments, generating the second AI model 130B may include updating the first AI model 130A to a configuration of the second AI model 130B. In other embodiments, the second AI model 130B may replace the first AI model 130A.

The processing from 308 proceeds to 312 where sensor data from at least one or more non-image sensors and/or text data are received. In some embodiments, the data is received in one of the programs 124C that may generate the second AI model 130B. In other embodiments, the data is received in one or more other devices that train the second AI model 130B. This data is used to train the second AI model 130B or update the first AI model 130A to the second AI model 130B. In some embodiments, the second AI model 130B may be the same as the first AI model 130A, but trained using the data described herein. Accordingly, the second AI model 130B is trained to characterize items that are different than items the first AI model 130A is trained to characterize. In some embodiments, the data used to train the second AI model 130B includes at least some of the data used to train the first AI model 130A, so the second AI model 130B may characterize at least some of the items that the first AI model 130A was trained to characterize. In some embodiments, a user of the system 100 may be prompted to train the second AI model 130B. The user may then initiate the training such as by the CIM 126 (FIG. 1). In other embodiments, the user may initiate training of the second AI model 130B described herein without being prompted.

The non-image sensors may include, for example, temperature sensors, acoustic sensors, humidity sensors, liquid volumes sensors, vibration sensors, current sensors, and other sensors related to the operation of the system 100. The text data may include tests being performed (e.g., assay types), patient information (e.g., age, symptoms, etc.), date of the test, time of the test, system logs (e.g., system status), label information from the specimen containers 102 (e.g., data from the label 222—FIGS. 2A-2B), and other data related to tests being performed by the system 100.

The method 300 may proceed to 314 where the second AI model 130B (FIG. 1) is trained using the captured image and data generated by at least one of the non-image sensors or at least some of the text data. As described above, in some embodiments, the second AI model 130B is trained using at least some of the data used to train the first AI model 130A so the second AI model 130B is capable of characterizing images on which the first AI model 130A is trained. In some embodiments, a plurality of images having characterization confidences below the pre-established threshold are stored and used to train the second AI model 130B. For example, the images having characterization confidences below the pre-established confidence may be stored in the memory 124B, the cloud, and/or a fixed storage and used collectively to train the second AI model 130B in 314.

In some embodiments, updating the first AI model 130A may include updating the model capacity (e.g., adding residual layers) or model weights. The model weights determine which data samples are used for backpropagation by the AI model. In some embodiments, the AI model may include a deep network, such as a variational auto-encoder, that can be trained to determine if data provided is out of a training manifold or within an original training manifold. In embodiments where the second AI model 130B replaces the first AI model 130A, the second AI model 130B may be trained as described above.

The data used to train the second AI model 130B may be referred to as the sampling data. The sampling data incorporated into the second AI model 130B may be selected to avoid divergence of the second AI model 130B. In divergence, the second AI model 130B will perform worse than the first AI model 130A that was trained on the first validation dataset, which may be a gold standard or ground truth. Divergence may be indicative as either underfitting or “catastrophic forgetting.” Underfitting may be identified by the second AI model 130B not able to identify or characterize items in the new data. Catastrophic forgetting may be identified as the second AI model 130B overfitted to the new data, wherein the second AI model 130B is not able to characterize items in in the first validation dataset. Neither underfitting nor catastrophic forgetting may be acceptable because underfitting restricts the range of improvements that can be made and catastrophic forgetting (e.g., overfitting) may no longer meet the requirements of the regulatory clearance obtained based on the first validation dataset.

Based on the foregoing, in some embodiments, the first AI model 130A may only be updated when the updates are likely to help the system 100. In some embodiments, outliers may exist in the sample data that may cause the second AI model to degenerate, such as by underfitting or catastrophic forgetting as described above. The problems may be avoided by having access to a validation dataset on which the performance of the second AI model 130B may be evaluated and if a divergence occurs, the second AI model 130B may be rollbacked to an older AI model, such as the first AI model 130A. In some embodiments, the first AI model 130A may be updated continuously.

In some embodiments, training the second AI model 130B may include validating the second AI model 130B using a validation dataset as described above. The validation dataset may be data correlating the captured images to certain characterizations. In other embodiments, the validation dataset may be based on data received from other sources, such as other systems or data sets generated specifically to validate the second AI model 130B.

The second AI model 130B may be validated in 316. For example, the captured images and/or other images having characteristics similar to the captured images may be characterized using the second AI model 130B. Characterization confidences performed using the second AI model 130B may be determined. If the characterization confidences are below a pre-selected threshold, the second AI model 130B may not be trained correctly. In such situations, the second AI model 130B may be trained further or replaced with the first AI model 130A. If the characterization confidences are greater than the pre-selected threshold, the method 300 may proceed to 318 where the second AI model 130B is used to characterize images as described herein.

The method 300 will now be described implemented in modules 108 and/or the instruments 110 of the system 100 (FIG. 1). In some embodiments, one or more of the modules 108 may be implemented as a quality check module 132 that may be located adjacent the track 112. The quality check module 132 may be configured to capture one or more images of the specimen containers 102 and/or the specimens (e.g., specimen 216, FIGS. 2A-2B) located therein, wherein the one or more images comprise image data. The computer 124 or other device may analyze the image data using the first AI model 130A or the second AI model 130B to determine whether the specimen is in proper condition for analysis by the modules 108 and/or the instruments 110. The analysis may further determine the type of test or analysis in which the specimen container 102 is configured.

Additional reference is made to FIGS. 4A and 4B, which illustrate an embodiment of the quality check module 132 configured to perform at least one of the characterizations described herein. FIG. 4A illustrates a schematic top view of the quality check module 132 with a top removed and FIG. 4B illustrates a schematic side view of the quality check module 132 of FIG. 4A.

The quality check module 132 may include a housing (not shown) that may at least partially surround or cover the track 112 to minimize outside lighting influences. The specimen container 202 may be located inside the housing at an imaging location 442 during the image-capturing sequences. The housing may include one or more doors (not shown) to allow the carrier 214 to enter into and/or exit from the quality check module 132. In some embodiments, a ceiling (not shown) of the housing may include an opening that allows the specimen container 202 to be loaded into the carrier 214 by a robot (not shown).

The quality check module 132 may include one or more imaging devices 440. The imaging devices 440 are referred to individually as a first imaging device 440A, a second imaging device 440B, and a third imaging device 440C. The imaging devices 440 may be configured to capture images of the specimen container 202 and specimen 216 at the imaging location 442 from multiple viewpoints (e.g., viewpoints labeled 1, 2, and 3). While three imaging devices 440 are shown in FIG. 4A, optionally, two, four, or more imaging devices can be used. The viewpoints 1-3 may be arranged so that they are approximately equally spaced from one another, such as about 120° radially from one another, as shown. The images may be captured in a round robin fashion, for example, one or more images from viewpoint 1 may be captured followed sequentially by capturing images from viewpoints 2 and 3. Other sequences of capturing images may be used. Other arrangements of the imaging devices 440 may be used.

The images of the specimen 216 and/or the specimen container 202 may be captured while the specimen container 202 is residing in the carrier 214 at the imaging location 442. The field of view of the multiple images obtained by the imaging devices 440 may overlap slightly in a circumferential extent. Thus, in some embodiments, portions of the images may be digitally added to arrive at a complete image of the serum or plasma portion 216SP (FIG. 2A) for analysis. Each of the imaging devices 440 may be triggered by triggering signals provided on communication lines 443A, 443B, 443C generated by the computer 124. Each of the captured images may be processed by the computer 124 according to one or more embodiments described herein.

The imaging devices 440 may be any suitable devices configured to capture digital images. In some embodiments, each of the imaging devices 440 may be a conventional digital camera capable of capturing pixelated images, a charged coupled device (CCD), an array of photodetectors, one or more CMOS sensors, or the like. The sizes of the captured images may be about 2560×694 pixels, for example. In other embodiments, the imaging devices 440 may capture images having sizes of about 1280×387 pixels, for example. The captured images may have other sizes.

The quality check module 132 may include one or more light sources 444 that are configured to illuminate the specimen container 202 and/or the specimen 216 during image capturing. In the embodiment of FIG. 4A, the quality check module 132 includes three light sources 444, which are referred to individually as a first light source 444A, a second light source 444B, and a third light source 444C. In some embodiments, the quality check module 132 may provide front lighting of the imaging location 442.

In addition to the imaging devices 440, the quality check module 132 may include one or more non-image sensors. Non-image sensors are sensors that may be used by a module, such as the quality check module 132 to generate data, other than image data, related to the operation of the module. Image data is data representative of a captured image and may include a plurality pixel values. Instruments also may include non-image sensors. The embodiment of the quality check module 132 depicted in FIGS. 4A-4B includes five non-image sensors. The non-image sensors include a current sensor 450, a vibration sensor 452, a humidity sensor 454, a temperature sensor 456, and an acoustic sensor 458. Other embodiments of the modules 108 and the instruments 110 include more or fewer non-image sensors.

The current sensor 450 may measure current drawn by one or more components of a module, such as the quality check module 132, and generate current data. In the embodiment of FIGS. 4A-4B, the current sensor 450 is configured to measure current drawn by a motor 460. The motor 460 may move one or more items in the quality check module 132. In the embodiment depicted in FIGS. 4A-4B, the track 112 may be movable by way of the motor 460. Accordingly, the current sensor 450 may measure current drawn by the motor 460. Current data (e.g., measured current) generated by the current sensor 450 may be transmitted to the computer 124. In some embodiments, the current data may be used to train the second AI model 130B (FIG. 1). Current data may be generated by other devices that draw current.

The vibration sensor 452 may measure vibration of a module, such as the quality check module 132, and/or one or more components in the module and generate vibration data. In the embodiment of FIGS. 4A-4B, the vibration sensor 452 is configured to measure vibration of the track 112, which may affect the specimen 216 in the specimen container 202. Vibration data (e.g., measured vibration) generated by the vibration sensor 452 may be transmitted to the computer 124. In some embodiments, the vibration data may be used to train the second AI model 130B (FIG. 1). Vibration data may be generated by other sources.

The humidity sensor 454 may measure humidity in a module, such as the quality check module 132, and generate humidity data. In some embodiments, the humidity sensor 454 may measure humidity in the location of the system 100 (FIG. 1) and not in any specific module. The humidity data may be transmitted to the computer 124 where the humidity data may be used train the second AI model 130B (FIG. 1).

The temperature sensor 456 may measure temperature in a module, such as the quality check module 132, and generate temperature data. In some embodiments, the temperature sensor 456 may measure temperature of a component within a module or within the system 100 (FIG. 1). In some embodiments, the temperature sensor 456 may measure ambient air temperature in a module or proximate the system 100. The temperature data may be transmitted to the computer 124 where the temperature data may be used to train the second AI model 130B (FIG. 1).

The acoustic sensor 458 may measure noise in a module, such as the quality check module 132, and may generate acoustic data. In some embodiments, the acoustic sensor 458 may measure ambient noise proximate the system 100 (FIG. 1). The noise may be generated by a component that is vibrating and may affect the specimen 216. The acoustic data may be transmitted to the computer 124 where the acoustic data may be used to train the second AI model 130B (FIG. 1).

In some embodiments, the characterizations associated with data generated by sensors in the quality check module 132 may include determining a presence of and/or an extent or degree of hemolysis (H), icterus (I), and/or lipemia (L) contained in the specimen 216. In some embodiments, the characterization may determine whether the specimen 216 is normal (N). If the specimen 216 is found to contain low amounts of H, I and/or L, so as to be considered normal (N), the specimen 216 may continue on the track 112 where the specimen 216 may be analyzed (e.g., tested) by the one or more of the modules 108 and/or the instruments 110. Other pre-processing operations may be conducted on the specimen 216 and/or the specimen container 202.

In some embodiments, in addition to detection of HILN, the characterization may include segmentation of the specimen container 202 and/or the specimen 216. From the segmentation data, post processing may be used for quantification of the specimen 216 (i.e., determination of HSP, HSB, HTOT, HG, W, and/or possibly a determination of location of TC, LA, SG, and/or BG). In some embodiments, characterization of the physical attributes (e.g., size-height and/or width) of the specimen container 202 may be performed at the quality check module 132. From these characterizations, the size of the specimen container 202 may be calculated. Moreover, in some embodiments, the quality check module 132 may also determine the type of the cap 220 (FIGS. 2A-2B), which may be used as a safety check and may catch whether a wrong tube type has been used for the test or tests ordered.

Additional reference is made to FIG. 5, which illustrates a HILN network architecture 500 configured to perform one or more of the characterizations described herein. The HILN network architecture 500 may be implemented to operate and/or process data generated by the quality check module 132 (FIGS. 4A-4B) and other data sources, modules, and instruments as described herein. The HILN network architecture 500 includes an HILN network 502 and a database 504 that may be implemented in or accessible by the computer 124 (FIG. 1). The HILN network 502 may be at least partially implemented by an AI model, which may be the first AI model 130A or the second AI model 130B. In some embodiments, the first AI model 130A may be continuously updated or retrained to the second AI model 130B as described herein. The second AI model 130B may refer to the latest version of the AI model being used for characterization. Thus, the second AI model 130B may refer to the most up-to-date version of the AI model used for characterization.

In some embodiments, the database 504 may be resident in the memory 124B (FIG. 1) of the computer 124. In other embodiments, the database 504 may be a cloud-based database and may be accessible via the Internet, for example, or via a remote server/computer (not shown) that is separate from computer 124. The HILN network architecture 500 may provide feedback of training updates, such as training updates 508 that may be continuous to the HILN network 502 by way of information stored in the database 504.

Data used for the training updates 508 may be stored in the database 504. In some embodiments, the data may be stored in different databases that are collectively referred to as the database 504. The database may include non-image data 510 from the non-image sensors and text data 512. In some embodiments, the non-image data 510 may be generated at the same time the image data is generated. Likewise, the text data 512 may correlate to the image data. For example, the text data 512 may be related to specimens and/or specimen containers captured to generate the image data. The data used for the for the training updates 508 may also include original data used to train the first AI model 130A. Accordingly, the updated AI model (the second AI model 130B) may also be configured to characterize images that the first AI model 130A was trained to characterize.

In some embodiments, the database 504 may include image data 514 of images having low characterization confidences. For example, referring to FIG. 3, if the characterization confidence of an image is not above the pre-selected characterization confidence in 308, the image data of the low confidence characterization image may be used to train the second AI model 130B as described in 316. This image data may be passed from the HILN network 502 to the database 504. As described above, the non-image data 510 and/or the text data 512 corresponding to these images may also be used to train the second AI model 130B as described herein.

The training updates 508 may access the database 504 to obtain data to train the AI model(s) on a regular basis, upon detection of low characterization confidence, or by an action of a user. In some embodiments, the training updates 508 may be applied immediately into the second AI model 130B. In such embodiments, the training updates 508 may be performed as data is received into the database 504. In some embodiments, a user of the system 100 (FIG. 1) such as the quality check module 132 may input data into the CIM 126 (FIG. 1), for example, that causes the training updates 508 to commence. For example, the first AI model 130A may be retrained in response to an input by the user.

In some embodiments, the training updates 508 may be initiated automatically, such as without user initiation. For example, the HILN network 502 or other component may determine the characterization confidences. If the characterization confidences are below a pre-established threshold, such as the pre-established threshold in 308 of FIG. 3, then the computer 124 (FIG. 1) or another device may initiate the training updates 508 as described herein.

In some embodiments, the training updates 508 may be performed locally, such as in the computer 124 (FIG. 1) or the training updates 508 may be performed remotely and then downloaded to the HILN network 502. The second AI model 130B may be tested (validated) on verification/validation data (e.g., data that was used for regulatory approval) in addition to newly collected data that may verify/validate the image data having the low characterization confidences. The training updates 508 may be performed without interrupting the existing workflow in the system 100 (FIG. 1).

In some embodiments, the HILN network 502 or a program receiving data from the HILN network 502 may generate a performance report related to the second AI model 130B. In some embodiments, the performance report may be in compliance with a regulatory process and may expedite regulatory approval of the updated HILN network 502. In some embodiments, the performance report may highlight improvement of the second AI model 130B over the first AI model 130A.

The HILN network architecture 500 may be implemented in a quality check module 132 and controlled by the computer 124 (FIG. 1), such as by the programs 124C. During operation of the quality check module 132 implementing the HILN network architecture 500, the specimen container 202 (FIGS. 2A-2B) may be provided at the imaging location 442 (FIGS. 4A and 4B) of the quality check module 132 as represented by functional block 520. Providing the specimen container 202 at imaging location 442 may be accomplished by stopping the carrier 214 (FIG. 2B) containing the specimen container 202 at the imaging location 442. Other methods of placing the specimen container 202 at the imaging location 442 may be used, such as placement by a robot (not shown).

One or more images (e.g., multi-viewpoint images) may be captured by at least one of the plurality of imaging devices 440 as represented at functional block 522. The raw image data for each of the captured images may be processed and consolidated as described in US Pat. App. Pub. 2019/0041318 to Wissmann et al. titled “Methods And Apparatus For Imaging A Specimen Container and/or Specimen Using Multiple Exposures” to process the raw image data as represented at functional block 524. In some embodiments, the raw image data may include a plurality of optimally exposed and normalized image data sets (hereinafter “image data sets”) in functional block 524. The processing in functional block 524 may produce the image data 514 (i.e., pixel data). The image data of a captured image data set of the specimen 216 (and specimen container 202) may be provided as input to the HILN network 502 in the HILN network architecture 500 in accordance with one or more embodiments. In some embodiments, the image data 514 may be raw image data.

The HILN network 502 may be configured to perform characterizations, such as segmentation and/or HILN determinations, on the image data 514 using the first AI model 130A and/or the second AI model 130B. The first AI model 130A is used in situations where the first AI model 130A has not been updated to or replaced by the second AI model 130B. In some embodiments, the segmentations and HILN determinations may be accomplished by a segmentation convolutional neural network (SCNN). Other types of HILN networks may be employed to provide segmentation and/or HILN determinations.

In some embodiments, the HILN network architecture 500 may perform pixel-level classification and may provide a detailed characterization of one or more of the captured images. The detailed characterization may include separation of the specimen container 202 from the background and a determination of a location and content of the serum or plasma portion 216SP of the specimen 216. In some embodiments, the HILN network 502 can be operative to assign a classification index (e.g., HIL or N) to each pixel of the image based on local appearances of each pixel. Pixel index information can be further processed by the HILN network 502 to determine a final HILN classification index for each pixel.

In some embodiments, the classification index may include multiple serum classes, including an un-centrifuged class, a normal class, and multiple classes/subclasses. In some embodiments, the classification may include 21 serum classes, including an un-centrifuged class, a normal class, and 19 HIL classes/subclasses, as described in greater detail below.

One challenge to determining an appropriate HILN classification index for a specimen 216 undergoing pre-screening at the quality check module 132 may result from the small appearance differences within each sub-class of the H, I, and L classes. That is, the pixel data values of adjacent sub-classes can be very similar. To overcome these challenges, SCNN, which may be implemented by an AI model of the HILN network 502, may include a very deep semantic segmentation network (DSSN) that includes, in some embodiments, more than 100 operational layers.

To overcome appearance differences that may be caused by variations in specimen container type (e.g., size, shape, and/or type of glass or plastic material used in the container), the HILN network 502 may also include a container segmentation network (CSN) at the front end of the DSSN and implemented by the first AI model 130A or the second AI model 130B. The CSN may be configured to determine an output container type that may include, for example, a type of the specimen container 202 indicating height HT and width W (or diameter), and/or a type and/or color of the cap 220. In some embodiments, the CSN may have a similar network structure as the DSSN, but shallower (i.e., with fewer layers). The DSSN may be configured to determine output boundary segmentation information 520, which may include locations and pixel information of the serum or plasma portion 216SP, the settled blood portion 216SB, the gel separator 216G, the air 224, and the label 222.

The HILN determination of a specimen characterized by the HILN network 502 may be a classification index 528 that, in some embodiments, may include an un-centrifuged class 522U, a normal class 522N, a hemolytic class 522H, an icteric class 522I, and a lipemic class 522L. In some embodiments, the hemolytic class 522H may include sub-classes H0, H1, H2, H3, H4, H5, and H6. The icteric class 522I may include sub-classes I0, I1, I2, I3, I4, I5, and I6. The lipemic class 522L may include sub-classes L0, L1, L2, L3, and L4. Each of the hemolytic class 522H, the icteric class 522I, and/or the lipemic class 522L may have, in some embodiments, other numbers of fine-grained sub-classes.

The captured images, the non-image data 510, and the text data 512 of imaging having incorrect or low confidence characterizations may be forwarded (and encrypted in some embodiments) to the database 504. Various algorithms and/or techniques may be used to identify incorrect characterizations and/or determine characterization confidences (i.e., predictions of the accuracy of characterization determinations). In some embodiments, the incorrect or low confidence determinations are determinations that the HILN determinations are incorrect or have low probabilities of being correct. Incorrect characterizations, as used herein, mean that the HILN determinations are improper because the corresponding characterization confidences are low. In embodiments where there is a low characterization confidence determination of the HILN class or class index, the low characterization confidence may be based on using a characterization confidence level of less than 0.9, for example. This characterization confidence level limit may be pre-selected by a user or determined based on regulatory requirements.

In some embodiments, the incorrect or low characterization confidence determination is a determination that the segmentation determination is incorrect or has low confidence. In particular, the incorrect determination of the segmentation may involve identification of a region, such as the serum or plasma portion 216SP, the gel separator 216G, or the settled blood portion 216SB that has low characterization confidence or that is not in certain order with respect to one or more other regions. For example, the cap 220 may be expected to be on top of the serum or plasma portion 216SP and the gel separator 216G may be below the serum or plasma portion 216SP. If the relative positioning is not met, then an error may have occurred during the segmentation.

A determination of low characterization confidence in the segmentation or other process may involve reviewing a probability score for each segmented pixel (or collection of pixels in a superpixel—e.g., a collection of pixels, such as 11 pixels). If the probability score indicating a particular classification (e.g., serum or plasma portion 216SP) of a region of pixels has too much disagreement, then that segmented pixel would likely be a candidate for low characterization confidence. The probability scores of the pixels (or superpixels) of a region that has been segmented (e.g., region classified at serum or plasma portion 216SP) can be aggregated to determine if the region contains too much disagreement. In this case, the region would likely be a candidate for a low characterization confidence if the characterization confidence level is less than the pre-selected value (e.g., 0.9). Other suitable aggregated characterization confidence levels for a region can be used.

Based on the incorrect or low characterization confidences, the computer 124 (FIG. 1) may initiate the training updates 508 (e.g., additional training images) automatically and/or in conjunction with user input. In some embodiments, the training updates 508 may be manually annotating the images that have low characterization confidences. Manual annotation of the images may include graphically outlining at least the serum or plasma portion 216SP in the respective training image and/or the settled blood portion 216SB, the air 224, the cap 220, the label 222, and the gel separator 214G). Manual annotation of the training images can include assigning an HILN classification and may also include assigning an index value to the serum or plasma portion 216SP. In other embodiments, the training updates 508 may be provided with automatically generated annotations of the images of the specimen characterizations determined to be incorrect or having low characterization confidences. The automatic annotation can be provided by semi-supervised approach, boot-strapping, or using a pre-trained segmentation/classification algorithm, for example.

The training updates 508 may be incorporated into the HILN network 502 (e.g., via the Internet or a physical media). In some embodiments, the training updates 508 may be an update applied to the first AI model 130A and in other embodiments, the training updates 508 may generate a new AI model (e.g., the second AI model 130B) as described herein. The incorporation of the training updates 508 into the HILN network 502 may be automatic under the control of the computer 124 (FIG. 1) or by a prompting via the CIM 126 (of FIG. 1). The second AI model 130B model may be tested (e.g., validated) on the verification/validation data that was used for regulatory approval in addition to the newly collected image data 514, the non-image data 510, and/or the text data 512. The computer 124 can then generate a performance report in compliance with performance criteria, such as from a regulatory process, wherein it can highlight the improvement over the first AI model 130A. Based on this performance criteria, a user can approve the update. These updates can happen without interrupting the existing workflow. In some embodiments, the updates can be performed by a service technician or it can happen over remotely over the Internet.

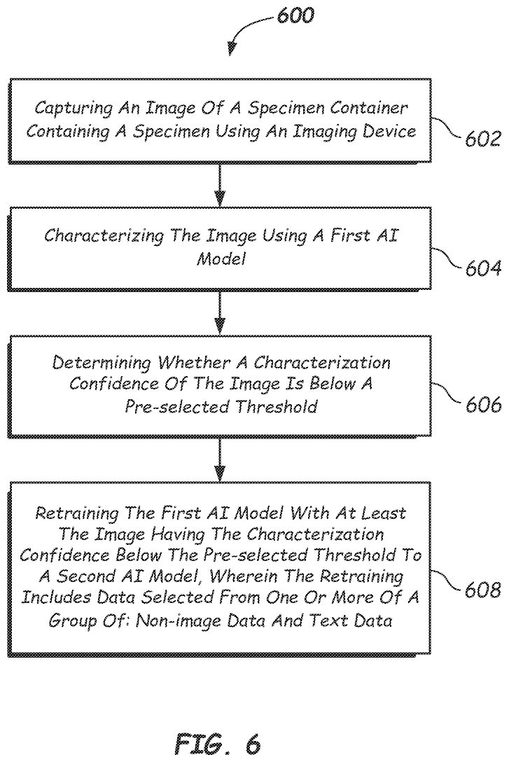

Additional reference is made to FIG. 6, which illustrates a flowchart showing a method 600 of characterizing a specimen container (e.g., specimen container 202) or a specimen (e.g., specimen 216) in an automated diagnostic system (e.g., automated diagnostic system 100). The method 600 includes, in 602, capturing an image of a specimen container containing a specimen using an imaging device (e.g., one or more of the imaging devices 440). The method 600 includes, in 604, characterizing the image using a first AI model (e.g., first AI model 130A). The method 600 includes, in 606, determining whether a characterization confidence of the image is below a pre-selected threshold. The method 600 includes, in 608, retraining the first AI model with at least the image having the characterization confidence below the pre-selected threshold to a second AI model (e.g., second AI model 130B), wherein the retraining includes data selected from one or more of a group of: non-image data (e.g., non-image data 510), and text data (e.g., text data 512).

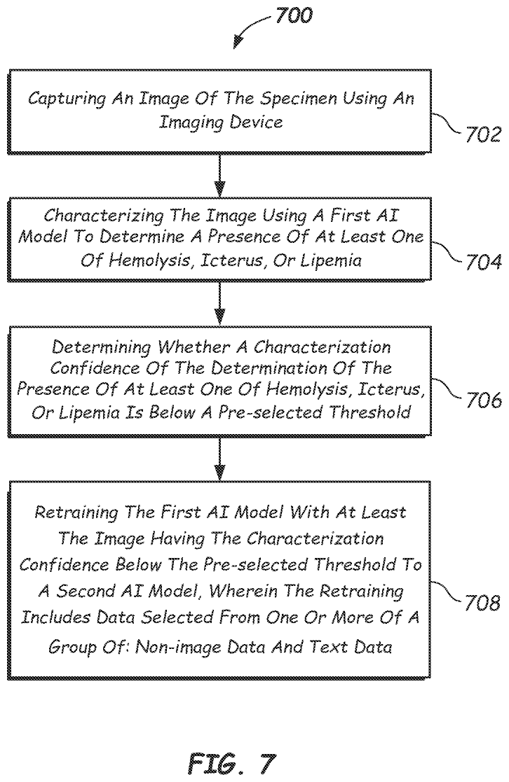

Additional reference is made to FIG. 7, which illustrates a flowchart showing a method 700 of characterizing a specimen (e.g., specimen 216) in an automated diagnostic system (e.g., automated diagnostic system 100). The method 700 includes, in 702, capturing an image of the specimen (e.g., specimen 216) using an imaging device (e.g., one or more of the imaging devices 440). The method 700 includes, in 704, characterizing the image using a first AI model to determine a presence of at least one of hemolysis, icterus, or lipemia. The method 700 includes, in 706, determining whether a characterization confidence of the determination the presence of at least one of hemolysis, icterus, or lipemia is below a pre-selected threshold. The method 700 includes, in 708, retraining the first AI model with at least the image having the characterization confidence below the pre-selected threshold to a second AI model (e.g., second AI model 130B), wherein the retraining includes data selected from one or more of a group of: non-image data (e.g., non-image data 510), and text data (e.g., text data 512).

While the disclosure is susceptible to various modifications and alternative forms, specific method and apparatus embodiments have been shown by way of example in the drawings and are described in detail herein. It should be understood, however, that the particular methods and apparatus disclosed herein are not intended to limit the disclosure but, to the contrary, to cover all modifications, equivalents, and alternatives falling within the scope of the claims.

What is claimed is:

1. A method of characterizing a specimen container or a specimen in an automated diagnostic system, comprising:
capturing an image of a specimen container containing a specimen using an imaging device;
characterizing the image using a first artificial intelligence (AI) model;
determining whether a characterization confidence of the image is below a pre-selected threshold; and
retraining the first AI model with at least the image having the characterization confidence below the pre-selected threshold to a second AI model, wherein the retraining includes data selected from one or more of a group of:
non-image data, and
text data.

2. The method of claim 1, comprising validating that the second AI model performs the characterizing with a characterization confidence above a pre-established threshold.

3. The method of claim 1, comprising validating the second AI model on a validation dataset.

4. The method of claim 1, comprising:
storing the image having the characterization confidence below the pre-selected threshold in a database including one or more other images having characterization confidences below the pre-selected threshold; and
retraining the first AI model with one or more of the images stored in the database.

5. The method of claim 1, wherein retraining the first AI model comprises replacing the first AI model with the second AI model.

6. The method of claim 1, comprising training the first AI model on a validation dataset obtained from a plurality of automated diagnostic systems.

7. The method of claim 1, comprising receiving a user input, wherein the first AI model is retrained in response to the user input.

8. The method of claim 1, wherein the characterizing comprises determining a presence of at least one of hemolysis, icterus, or lipemia in a serum or plasma portion of the specimen.

9. The method of claim 1, wherein the characterizing comprises determining at least one of an index of hemolysis, an index of icterus, or an index of lipemia of a serum or plasma portion of the specimen.

10. The method of claim 1, wherein the characterizing comprises segmenting the specimen.

11. The method of claim 1, wherein the characterizing comprises segmenting the specimen to identify at least a serum or plasma portion and a settled blood portion.

12. The method of claim 11, comprising determining a height of at least one of the serum or plasma portion or the settled blood portion.

13. The method of claim 1, wherein the characterizing comprises determining whether a cap is present on the specimen container.

14. The method of claim 1, wherein the characterizing comprises determining a color of a cap on the specimen container.

15. The method of claim 1, wherein the characterizing comprises determining a type of a cap on the specimen container.

16. The method of claim 1, wherein identifying if the characterization confidence is below the pre-selected threshold comprises identifying if the characterization confidence is less than 0.9 in a range between 0.0 and 1.0.

17. The method of claim 1, wherein identifying if the characterization confidence is below a pre-selected threshold comprises identifying if the characterization confidence is less than 0.8 in a range between 0.0 and 1.0.

18. The method of claim 1, comprising segmenting an image of the specimen or the specimen container, wherein identifying if the characterization confidence is below a pre-selected threshold comprises determining if less than 90 percent of pixel values in a segment are classified the same.

19. The method of claim 1, wherein the non-image data includes temperature data.

20. The method of claim 1, wherein the non-image data includes humidity data.

21. The method of claim 1, wherein the non-image data includes at least one of vibration data, current data, and acoustic data.

22. The method of claim 1, wherein the text data includes information related to a person from whom the specimen was taken.

23. A method of characterizing a specimen in an automated diagnostic system, comprising:
capturing an image of the specimen using an imaging device;
characterizing the image using a first artificial intelligence (AI) model to determine a presence of at least one of hemolysis, icterus, or lipemia;
determining whether a characterization confidence of the determination the presence of at least one of hemolysis, icterus, or lipemia is below a pre-selected threshold; and
retraining the first AI model with at least the image having the characterization confidence below the pre-selected threshold to a second AI model, wherein the retraining includes data selected from one or more of a group of:
non-image data, and
text data.

24. The method of claim 23, wherein the non-image data includes at least one of temperature data, humidity data, vibration data, current data, and acoustic data.

25. The method of claim 23, wherein the text data includes information related to a person from whom the specimen was taken.

26. An automated diagnostic system, comprising:
an imaging device configured to capture an image of a specimen container containing a specimen; and
a computer configured to:
characterize the image using a first artificial intelligence (AI) model;
determining whether a characterization confidence of the image is below a pre-selected threshold; and
retrain the first AI model with at least the image having the characterization confidence below the pre-selected threshold to a second AI model, wherein the retraining includes data selected from one or more of a group of:
non-image data, and
text data.
